(19) United States
(12) Patent Application Publication
(10) Pub. No.: US 2007/0148035 A1
(43) Pub. Date: Jun. 28, 2007

(54) Process for DNA decontamination

(76) Inventors: Armin Tgetgel, Huglfing (DE); Joachim Siedel, Bernried (DE); Alois Rainer, Munchen (DE); Wolf-Dieter Engel, Feldafing (DE)

Correspondence Address: ROCHE DIAGNOSTICS OPERATIONS INC., 9115 Hague Road, Indianapolis, IN, 46250-0457, US

(21) Appl. No.: 11/564,441

(22) Filed: Nov. 29, 2006

Related U.S. Application Data
(60) Provisional application No. 60/741,094, filed on Nov. 30, 2005.

(51) Int. Cl. C12M 1/34; A61L 2/20

(52) U.S. Cl. 422/34; 435/287.2

(57) ABSTRACT

A method and a disposable to avoid false nucleic acid amplification results based on the alklylation of DNA present at surfaces of disposables. More particularly, the present invention is directed to an improved method and a disposable, whereby the alkylation of DNA is performed using ethylene oxide.

### RELATED APPLICATIONS

This application claims priority to provisional application 60/741,094 filed Nov. 30, 2005.

### FIELD OF THE INVENTION

The present invention provides a method to obtain DNA molecules at surfaces of disposable devices or labware in an unamplificable form as well as disposable devices or labware that are free of amplifiable DNA.

### BACKGROUND OF THE INVENTION

Biological contamination is a common problem in many fields of science, health care and industry. Especially for the food industry and for medical services the absence of potentially pathogenic microorganisms such as bacteria, viruses, spores, protozoa and fungi on devices has to be ascertained, even though an environmental completely free of such organisms is not achievable. With respect to analytical science such biological contaminations may have drastic effects on measurement leading to false results.

There are many techniques known to someone skilled in the art that are suitable for the sterilization of devices, whereas the proper method depends on the material of the device and the required extent of sterility. Known sterilization methods are e.g. electromagnetic radiation, steam, hot air, hydrogen peroxide plasma or treatment with certain other chemicals.

All sterilization methods having in common that the pathogenic organisms are altered in such a way that reproduction is no longer possible. However, for devices and disposable articles or items that are used for analysis of nucleic acids, in particular for PCR applications, the mere sterility is not enough, because even dead pathogenic organisms may still have amplifiable DNA or RNA molecules.

A well known technique for the sterilization of thermal unstable materials comprises the use of ethylene oxide, because this substance is effective even at temperatures below 10° C. Because ethylene oxide is gaseous at room temperature and atmospheric pressure, the substance can reach even devices that are surrounded by certain packing materials.

Ethylene oxide is known to act with biological material through alkylation producing irreversible damage to all components of the pathogenic organisms like nucleic acids, proteins and enzymes and therefore, further reproduction is prevent (sterilization).

Nevertheless, it has not been demonstrated so far that ethylene oxide treatment does not only lead to a sterilization, but simultaneously also enables for the complete inactivation (=decontamination) of nucleic acid (DNA,RNA), even if said nucleic acid is still contained within e.g. microbial organisms.

Within the present invention it was found that ethylene oxide treatment of disposable articles or items can be performed in such an effective way that no amplifiable nucleic acid contamination remains and that said disposable articles or items are therefore applicable even for highly sensitive PCR analytics.

### SUMMARY OF THE INVENTION

The present invention comprises a method to alkylate DNA molecules at surfaces of disposable articles or items comprising providing said disposable in a temperature-controlled pressure chamber, adjusting the parameters of said temperature-controlled pressure chamber, gassing said disposable with ethylene oxide, rinsing said disposable with cleaning gas and desorbing of ethylene oxide. The invention further comprises disposable articles or items free of amplifiable DNA produced by gassing of said disposable articles or items with ethylene oxide according to the above method.

More particularly, the present invention comprises a method of treating a surface of an article to eliminate amplifiable nucleic acid molecules present on said surface, said method comprising:
sealing said article within an interior space of a temperature-controlled pressure chamber,
adjusting the pressure within the interior space of said chamber to about 40 to about 800 mbars,
increasing the temperature of the interior space to about 30° C. to about 60° C.,
introducing ethylene oxide into said interior space and in contact with said surface,
incubating the article in the present of the ethylene oxide at a pressure of about 40 to about 800 mbars and a temperature of about 30° C. to about 60° C. for a predetermined length of time,
rinsing said article with cleaning gas, and
desorbing the ethylene oxide.

The present invention also comprises a method of treating a surface of an article to eliminate amplifiable nucleic acid molecules present on said surface, said method comprising:
sealing said article within an interior space of a temperature-controlled pressure chamber,
humidifying the interior space to about 50% to about 90% relative humidity,
adjusting the pressure within the interior space of said chamber to about 40 to about 800 mbars,
introducing ethylene oxide into said interior space and in contact with said surface,
incubating the article in the presence of the ethylene oxide at a pressure of about 40 to about 800 mbars and about 50% to about 90% relative humidity for a predetermined length of time,
rinsing said article with a cleaning gas, and
desorbing the ethylene oxide.

### DESCRIPTION OF THE FIGURES

FIG. 1: PCR plot illustrating the alkylation efficiency of an ethylene oxide treatment of plastic disposables contaminated with spores.

FIG. 2: PCR plot illustrating the alkylation efficiency of an ethylene oxide treatment of glass disposables contaminated with spores.

FIG. 3: PCR plots comprising amplification curves performed with (a) and without ethylene oxide treatment (b) of disposables using a SybrGreen assay.

FIG. 4: PCR plots comprising amplification curves performed with (a) and without ethylene oxide treatment (b) of disposables using a hybridization probe assay.

### DESCRIPTION OF THE INVENTION

Many alternatives to avoid the amplification of nucleic acid contaminations are known to someone skilled in the art, but none of the alternatives are applicable to eliminate false nucleic acid amplification results originating from nucleic acid contaminations on surfaces of e.g. thermolabile disposables.

Therefore, the present invention is directed to a method and a disposable to avoid false nucleic acid amplification results based on the alkylation of DNA present at surfaces of disposables. More particular, the present invention is directed to an improved method and a disposable, whereas the alkylation of DNA is performed using ethylene oxide.

In addition to disposable articles or items, the method of the present invention is useful for treating the surface of any device, instrument, or labware, wherein labware includes any vessel for holding or transferring a fluid or solid. Thus labware includes pipettes, petri dishes, test tubes, tubing, and any other equipment intended to hold or transfer solutions comprising nucleic acids.

One subject matter of the present invention is a method to alkylate DNA molecules at surfaces of disposables comprising:
providing said disposable in a temperature-controlled pressure chamber,
adjusting the parameters of said temperature-controlled pressure chamber,
gassing said disposable with ethylene oxide,
rinsing said disposable with cleaning gas and
desorbing of ethylene oxide.

Throughout the present invention the alkylation of DNA means the chemical modification of DNA molecules by adding alkyl groups. Such an alkylation is known also for other cellular constituents of organisms, like proteins and enzymes. As a result of the alkylation of cellular constituents, cell reproduction is prevented and cell death ensues.

It is known that gaseous ethylene oxide (C₂H₄O) is a powerful alkylating agent for cellular constituents of organisms, if the correct conditions are provided. These include an adequate concentration of ethylene oxide and a necessary level of water within the organism. Since the process is a chemical reaction it is temperature dependent, whereas the reaction rate is increasing with temperature. These properties define the key characteristics of an ethylene oxide sterilization process.

The parameters of the ethylene oxide treatment can be controlled by using a temperature-controlled pressure chamber. Such a pressure chamber is preferably made out of stainless steel equipped with a vacuum pump to control the vacuum, a chamber jacket circulate hot liquid or air in order to adjust the temperature and a device to inject steam to the chamber affecting the humidity within the chamber.

After the gassing of disposables, ethylene oxide is adsorbed at the surface of the disposables as well as of the chamber itself. Due to the toxicity of ethylene oxide, it is necessary to remove the chemical prior to the removal of the disposables from the temperature-controlled pressure chamber. This is achieved by using a cleaning gas replacing the ethylene oxide atmosphere within the pressure chamber. Each replacement of the atmosphere within the pressure chamber by fresh cleaning gas is called a rinsing step within the present invention.

Note that mere sterility of devices and disposables may be insufficient for PCR application, because even dead pathogenic organisms may still have amplifiable DNA or RNA molecules causing false amplification results. Therefore, the parameters of the ethylene oxide treatment such as time, temperature, pressure, humidity and ethylene oxide concentration have to be adapted towards the sufficient suppression of DNA amplification.

In a method according to one embodiment of the present invention, said temperature-controlled pressure chamber is adjusted to 40-500 mbar and more than 15° C., preferably to between 45-55° C.

Prior to the gassing step air is removed from the pressure chamber using a vacuum pump to an initial pressure of about 40-500 mbar. Afterwards, ethylene oxide is injected to the chamber and consequently, the pressure within the chamber increases.

The temperature is an important parameter of the ethylene oxide treatment, because it is known that the rate of alkylation is strongly depending on the reaction temperature. Although ethylene oxide develops its alkylating power already at around 15° C., the necessary incubation time can be reduced to the sixth part, if the temperature is increased from 20 to 60° C. Note that the ethylene oxide starts to fluidify at temperatures between 10 and 15° C.

In another embodiment according to the present invention, said gassing of disposables is performed with a concentration of at least 100 g/cm³, preferably between 300-1200 g/cm³ at 400-800 mbar.

With respect to the concentration of the ethylene oxide two aspects have to be considered. On the one hand, the efficieny of DNA alkylation increases the increasing ethylene oxide concentration and therefore, high ethylene oxide concentrations are preferred. Note that this is only true up to a certain concentration, where a further increase in concentration no longer yields any improvement. On the other hand, ethylene oxide is toxic and therefore, increasing its amount adds higher health risks to the process.

In yet another embodiment according to the present invention, said gassing of disposables is performed for at least 1 hour, preferably between 3-8 hours.

The incubation time with the ethylene oxide is strongly depending on the concentration. For example, the incubation time for a standard sterilization is reduced from 460 to 280 min, if the concentration is increased from 200 to 600 mg/cm³.

Another embodiment of the method according to the present invention further comprises a humidification step prior to said gassing of disposables, whereas the humidity is adjusted to 50-90%.

Humidity is another important parameter of the ethylene oxide treatment, because it is known that ethylene oxide needs water to develop its alkylating power. Especially with respect to bacterial spores having a dry cellular wall, the effect of ethylene oxide is inhibited, if no humidity is added to the process. The humidity within the chamber is adjusted by inserting a controlled amount of steam.

In another embodiment according to the present invention, said humidity is adjusted incremental.

In this embodiment of the present invention, the final humidity within the pressure chamber is adjusted incremental performing several humidification steps. After each humidification step of inserting a controlled amount of steam, the atmosphere within the chamber may adjust during a waiting period of several minutes. This procedure helps to avoid droplet formation and supports a homogeneous humidity. It is preferred to adjust the humidity by more than 3 incremental steps.

In still another embodiment according to the present invention, said humidification step is performed for 5-35 minutes at 80-600 mbar.

After reaching the final humidity, a homogenous moisturization of the disposables is reached during an incubation time of between 5-35 minutes. During the humidification step, the pressure within the chamber is increased from the initial pressure to about 80-600 mbar due to the inserted steam.

In one embodiment of the method according to the present invention, said cleaning gas is air.

As mentioned before, the ethylene oxide has to be removed from the disposables and the temperature-controlled pressure chamber prior to the removal of the disposables. This is preferably performed by replacing the ethylene oxide atmosphere within the pressure chamber with air. The ethylene oxide itself is preferably converted to carbon dioxide via catalytic conversion step.

In another embodiment of the method according to the present invention, said rinsing is performed twice, preferably more than 3 times.

After the one or more rinsing steps, the disposables are finally stored at atmospheric pressure in order to desorb remaining ethylene oxide to an unperilous level.

In yet another embodiment of the method according to the present invention, said desorbing of ethylene oxide is performed at atmospheric pressure, preferably for at least 5 hours.

Note that the desorbing of ethylene oxide is strongly depending on the porosity and on the chemical composition of the disposable. In addition, it is known that ethylene oxide may form stable bonds with certain plastic materials and can not be adsorbed in this case at all. Therefore, it is possible to detect the presence of ethylene oxide at gassed disposables even after several weeks, but the amount of desorbing ethylene oxide is far below a harmful dose and has no influence on the subsequent PCR performance.

Another subject matter of the present invention is a disposable free amplifiable DNA produced by gassing of said disposables with ethylene oxide according to the method of the present invention.

Within the scope of the present invention all kinds of disposables may be turned into a disposable free of amplifiable DNA. Even thermolabile materials can be modified, because ethylene oxide develops its alkylating power already at around 15° C.

In one embodiment of the disposables according to the present invention, said disposables are products made of glass, plastic, ceramics or metal together with the respective packing materials of said products.

Note that even disposables already surrounded by packing material can be turned into a disposable free of amplifiable DNA. The only requirement for such a packing material is the permeability for humidity and ethylene oxide.

In another embodiment of the disposables according to the present invention, said disposables are surrounded by packing material, preferably said packing material is plastic or aluminum foil, paper and carton.

The following examples and figures are provided to aid the understanding of the present invention, the true scope of which is set forth in the appended claims. It is understood that modifications can be made in the procedures set forth without departing from the spirit of the invention.

### SPECIFIC EMBODIMENTS

### Example 1

### Gassing with Ethylene Oxide

The disposables were arranged in a 33 m³ stainless steel sterilization chamber and the chamber was set to initially 50 mbar and 48° C. Afterwards, the humidity of the chamber was incrementally (4 injections of steam) adjusted to 65%, whereas the pressure rose to 90 mbar. After a humidification time of 35 minutes, the chamber was filed with ethylene oxide (throughout the experiments gas with a purity of 98% was used) to a final concentration of 800 g/m³, whereas the pressure inside of the chamber rose to 500 mbar. After an incubation time with ethylene oxide of 4 hours, the chamber was rinsed 4 times with air. Finally, the disposables were stored for 7 days under constant ventilation in order to desorb the remaining ethylene oxide.

### Example 2

### Decontamination Efficieny at Plastic Disposables

To test the efficiency of the ethylene oxide treatment towards alkylation of nucleic acids for the purpose of avoiding amplification, a contamination was simulated by placing 10⁶ bacterial spores (Bacillus subtillis) on plastic disposables (polypropylene). Afterwards said plastic disposables were wrapped in plastic foil and treated with ethylene oxide as explained in example 1.

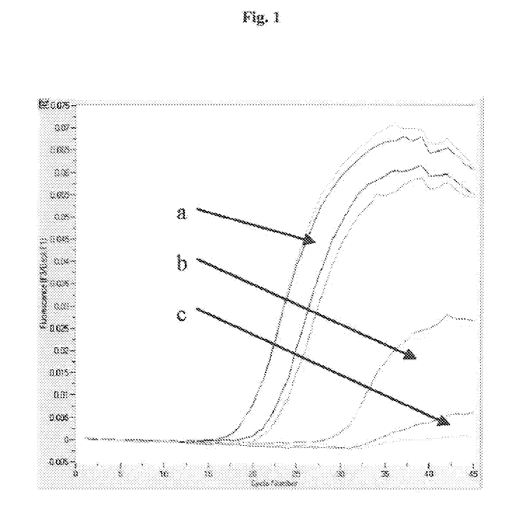

Then the plastic foil was removed and the ethylene oxide treated pathogenic organisms were eluted in 500 μl PCR-grade water. The eluat was then added directly into LIGHTCYCLER glass capillaries and a PCR amplification was performed using a LIGHTCYCLER 2.0 apparatus and the amplification kit LIGHTCYCLER FastStart DNA Master HybProbe (all of Roche Diagnostics GmbH; the quality of the kit reagents were improved in order to further remove traces of intrinsic DNA contamination). Note that the lysis and the DNA release from the bacterial spores occurs automatically during the hot start phase of the subsequent PCR. The primers of assay were designed to amplify a highly conserved region of the bacterial gene sequence coding for the 16s rRNA as known from the literature. To calculate the decontamination efficiency, an analogous sample without the ethylene oxide treatment was prepared and amplified as well. The amplification curves of this experiment are plotted in FIG. 1 (a: without ethylene oxide treatment, b: with ethylene oxide treatment, c: no-template-control) and a depletion factor of about 5000 was gained.

### Example 3

### Decontamination Efficiency at Glass Disposables

To test the efficieny of the ethylene oxide treatment towards alkylation of nucleic acids for the purpose of avoiding amplification, a contamination was simulated by placing 2×10⁶ bacterial spores (Bacillus subtillus) on glass disposables (100 μl capillaries for the LIGHTCYCLER 2.0, Roche Diagnostics GmbH). Afterwards said glass disposables were wrapped in plastic foil and treated with ethylene oxide as explained in example 1.

Then the plastic foil was removed and the ethylene oxide treated spores were eluted by adding PCR-grade water to the capillaries. Afterwards, a PCR amplification was performed in the same capillaries using a LIGHTCYCLER 2.0 apparatus and the amplification kit LIGHTCYCLER FastStart DNA Master HypProbe (both of Roche Diagnostics GmbH) analogous to Example 2. To verify the decontamination efficiency, additionally a sample without the ethylene oxide treatment as well as an ethylene oxide treated sample with subsequent addition of bacterial plasmids were prepared and amplified.

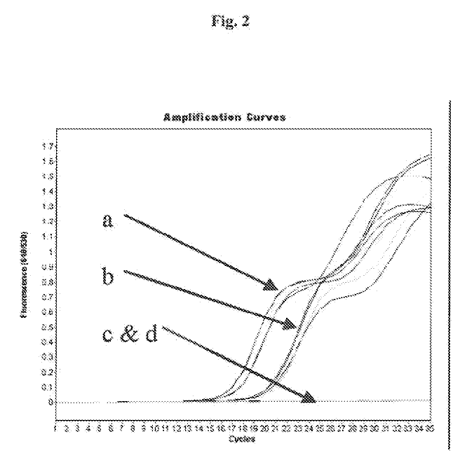

The amplification curves of this experiment are plotted in FIG. 2 (a: disposables without ethylene oxide treatment, b: disposables with ethylene oxide treatment and subsequent addition of 10⁴ copies of a Bacillus subtillis plasmid as reaction control, c: disposables with ethylene oxide treatment, d: no-template-control) and a decontamination efficiency of close to 100% was reached.

### Example 4: Impact of Ethylene Oxide on the PCR Performance

In order verify the influence of remaining ethylene oxide on the performance two different PCR assay were performed.

First, the influence of ethylene oxide was tested for a SybrGreen real-time PCR amplification of a dilution series (10⁵, 10⁴, 10³, 10² and 10 copies) of human house-keeping gene G-6-PDH (glucose-6-phosphate dehydrogenase) using the LIGHTCYCLER FastStart DNA Master SYBR Green I according to the specifications for the manufacturer (Roche Diagnostics GmbH) and a primer pair specific for the G-6-PDH gene. For this experiment the primers for the LIGHTCYCLER-t (9;22) Quantification Kit (Roche Diagnostics GmbH) were applied and a standard of the same kit was used to produce the dilution series of the house-keeping gene.

Second, the influence of ethylene oxide was tested for a real-time PCR amplification of a dilution series of a microbial plasmid ((10⁶, 10⁵, 10⁴, 10³, 10² 10 and 1 copies) using hybridization probes. For this experiment the LIGHTCYCLER-Parvovirus Quantification Kit (Roche Diagnostics GmbH) was used according to the specifications of the manufacturer, whereas for the dilution series of the microbial plasmid the standard of the kit was used.

For both experiments, untreated LIGHTCYCLER 480 multiwell plates with 384 well (Roche Diagnostics GmbH) were compared with multiwell plates treated with ethylene oxide (see Example 1) with respect to the respective PCR amplifications.

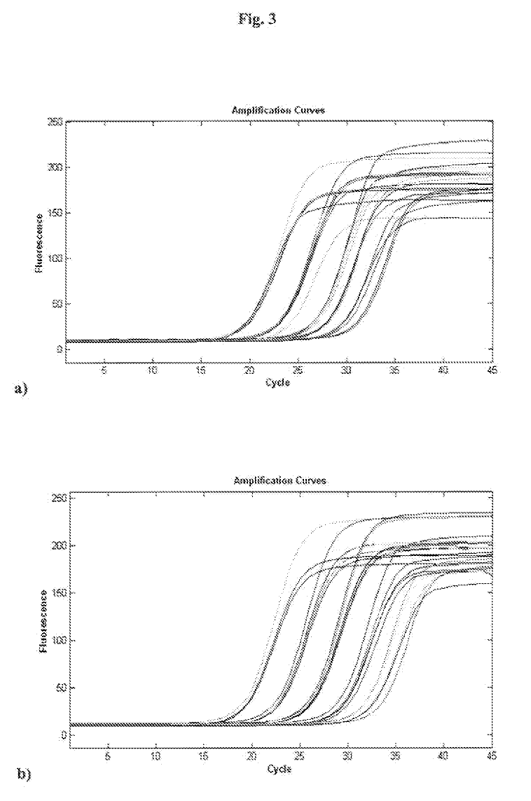

The amplification curves of the first experiment are plotted in FIG. 3 (a: treated with ethylene oxide, b: untreated). The following table comprises the cp-values of the experiment, indicating the no difference was detected in the subsequent PCR amplification.

| Copies | Treated | Untreated |
|---|---|---|
| 10⁵ | 19.54 | 19.03 |
| 10⁴ | 23.06 | 22.52 |
| 10³ | 26.77 | 25.89 |
| 10² | 28.68 | 25.97 |
| 10 | 30.19 | 29.16 |

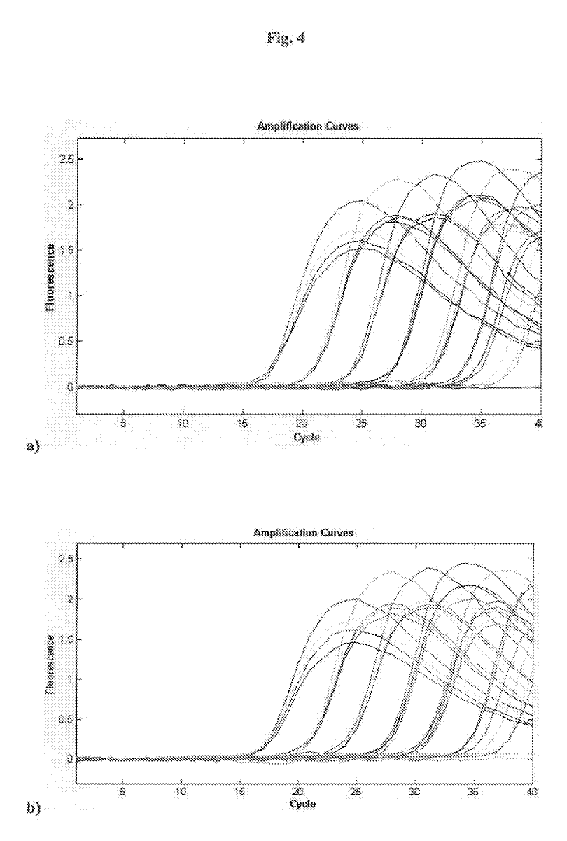

The amplification curves of the second experiment are plotted FIG. 4 (a: treated with ethylene oxide, b: untreated). The following table comprises the cp-values of the experiment, indicating that no difference was detected in the subsequent PCR amplification.

| Copies | Treated | Untreated |
|---|---|---|
| 10⁶ | 16.53 | 16.56 |
| 10⁵ | 20.17 | 20.16 |
| 10⁴ | 23.56 | 23.60 |
| 10³ | 27.15 | 27.01 |
| 10² | 30.18 | 30.02 |
| 10 | 33.53 | 33.76 |
| 1 | — | — |

What is claimed is:

1. A method of treating a surface of an article to eliminate amplifiable nucleic acid molecules present on said surface, said method comprising:
sealing said article within an interior space of a temperature-controlled pressure chamber,
adjusting the pressure within the interior space of said chamber to about 40 to about 800 mbars,
increasing the temperature of the interior space to about 30° C. to about 60° C.,
introducing ethylene oxide into said interior space and in contact with said surface,
incubating the article in the presence of the ethylene oxide at a pressure of about 40 to about 800 mbars and a temperature of about 30° C. to about 60° C. for predetermined length of time,
rinsing said article with a cleaning gas, and
desorbing the ethylene oxide.

2. The method according to claim 1 wherein the pressure is adjusted to about 40 to about 500 mbar and the temperature is adjusted to about 45° C. to about 55° C.

3. The method according to claim 1 wherein the ethylene oxide is introduced in an amount to provide a final concentration of at least 100 g/cm³.

4. The method according to claim 3 wherein the ethylene oxide is introduced in an amount to provide a final concentration of about 300 to about 1200 g/cm³.

5. The method according to claim 1 wherein said predetermined length of time is at least 1 hour.

6. The method according to claim 1 further comprising a step of humidifying the chamber prior to the step of introducing ethylene oxide into the chamber, wherein the interior space is adjusted to about 50% to about 90% relative humidity.

7. The method according to claim 6 wherein said humidifying step is performed for about 5 to about 35 minutes while maintaining the pressure of the interior space to about 80 to about 600 mbar.

8. The method according to claim 7 wherein the humidity of the chamber interior space is increased by incremental addition of steam in three or more separate steps to reach the desired final humidity.

9. The method according to claim 1 wherein said desorbing step is performed at atmospheric pressure.

10. The method according to claim 1 wherein the article is sealed within packaging prior to placing the article in the interior space, wherein said packaging is permeable to steam and ethylene oxide.

11. A method of treating a surface of an article of eliminate amplifiable nucleic acid molecules present on said surface, said method comprising:
sealing said article within an interior space of a temperature-controlled pressure chamber,
humidifying the interior space to about 50% to about 90% relative humidity,
adjusting the pressure within the interior space of said chamber to about 40 to about 800 mbars,
introducing ethylene oxide into said interior space and in contact with said surface,
incubating the article in presence of the ethylene oxide at a pressure of about 40 to about 800 mbars and about 50% to about 90% relative humidity for a predetermined length of time,
rinsing said article with a cleaning gas, and
desorbing the ethylene oxide.

12. The method according to claim 11 further comprising increasing the temperature of the interior space to about 30° C. to about 60° C. prior to said incubation step.

13. A device or labware that has been treated in accordance with the procedure of claim 1.

14. A device or labware free of amplifiable nucleic acids, the device or labware procedured by gassing of said device or labware with ethylene oxide according to claim 11.

15. The device or labware of claim 14, wherein said device or labware comprises glass, plastic, ceramics, or metal.

16. The device or labware of claim 15, wherein said device or labware is surrounded by packing material, said packing material selected from the group consisting of plastic, aluminum foil, paper, and carton.
